(19) United States
(12) Patent Application Publication
(10) Pub. No.: US 2014/0275872 A1
(43) Pub. Date: Sep. 18, 2014

(54) SYSTEMS AND METHODS FOR TESTING PATIENT MONITORS

(71) Applicant: Cercacor Laboratories, Inc., Irvine, CA (US)

(72) Inventors: Sean Merritt, Lake Forest, CA (US); Kevin Pauley, Lake Forest, CA (US); Jesse Chen, Lake Forest, CA (US); Justin Mathew Paul, Irvine, CA (US); Marcelo M. Lamego, Cupertino, CA (US)

(73) Assignee: Cercacor Laboratories, Inc., Irvine, CA (US)

(21) Appl. No.: 14/204,708

(22) Filed: Mar. 11, 2014

Related U.S. Application Data
(60) Provisional application No. 61/786,205, filed on Mar. 14, 2013.

(51) Int. Cl. A61B 5/1455

(52) U.S. Cl. CPC A61B 5/1455
USPC 600/322

(57) ABSTRACT

A quality control system for patient monitors is disclosed. The quality control system can include a quality check insert having optical properties. In an embodiment, the insert is placed within a sensor, irradiated with light, and then the light is detected after attenuation by the insert. The detected light is processed using the same or different processing methodologies typically used to determine measurement values for physiological parameters of a monitored patient. When a patient monitor is functioning properly, the results of the processing provide values within a predetermined range of values. When the patient monitor is not functioning properly, the results of the processing provide values outside the predetermined range of values. The quality control system can include quality control parameters indicative of a properly functioning active pulse motor of the sensor, emitters of the sensor, detectors of the sensor, accelerometers of the sensors, and/or temperature sensors of the system.

### CROSS-REFERENCE TO RELATED APPLICATIONS

This application claims the benefit under 35 U.S.C. §119(e) as a nonprovisional of U.S. Provisional Application No. 61/786,205, filed Mar. 14, 2013, titled SYSTEMS AND METHODS FOR TESTING PATIENT MONITORS, the entirety of which is incorporated herein by reference and made a part of this specification.

### BACKGROUND

1. Field of the Disclosure

The present disclosure relates to patient monitors, and in particular, relates to patient monitors that process signals indicative of light attenuated by body tissue carrying pulsing blood.

2. Description of the Related Art

In conventional noninvasive blood constituent measurements, such as blood oxygen saturation, light is transmitted at various wavelengths through a fleshy medium. Devices in this field generally include a light source(s) transmitting optical radiation into or reflecting off a measurement site, such as, body tissue carrying pulsing blood. A photodetection device(s) detects the attenuated light and outputs a detector signal(s) responsive to the detected attenuated light. A signal processing device(s) process the detector(s) signal(s) and outputs measurement values indicative of a blood constituents of interest, such as glucose, oxygen, methemoglobin, total hemoglobin, of other physiological parameters, or of other data or combinations of data useful in determining a state or trend of wellness of a patient. Patient monitoring systems are often adapted to position a finger proximate the light source and the light detector. For example, noninvasive sensors often include a clothespin-shaped housing that includes a contoured bed conforming generally to the shape of a finger. The housing substantially fixes the finger position with respect thereto, and therefore, positions the light source and detector proximate the finger to provide optical alignment through the finger.

Pulse oximetry utilizes a noninvasive sensor to measure oxygen saturation (SpO₂) and pulse rate. In general, the sensor has light emitting diodes (LEDs) that transmit optical radiation of red and infrared wavelengths into a tissue site and a detector that responds to the intensity of the optical radiation after attenuation. A processor processes the detector output to determine measurement values for SpO₂, pulse rate, and can output representative plethysmographic waveforms. Thus, “pulse oximetry” as used herein encompasses its broad ordinary meaning known to one of skill in the art, which includes at least those noninvasive procedures for measuring parameters of circulating blood. Moreover, “plethysmograph” as used herein (commonly referred to as “photoplethysmograph”), encompasses its broad ordinary meaning known to one of skill in the art, which includes at least data representative of volumetric changes within body tissue resulting from pulsing blood. Pulse oximeters capable of reading through motion induced noise are available from Masimo Corporation (“Masimo”) of Irvine, Calif. Moreover, portable and other oximeters capable of reading through motion induced noise are disclosed in at least U.S. Pat. Nos. 6,770,028, 6,658,276, 6,157,850, 6,002,952 5,769,785, and 5,758,644, the disclosure of which is hereby incorporated by reference in their entirety. Such reading through motion oximeters have gained rapid acceptance in a wide variety of medical applications, including surgical wards, intensive care and neonatal units, general wards, home care, physical training, and virtually all types of monitoring scenarios.

### SUMMARY

There is a need to noninvasively and accurately measure multiple physiological parameters. This disclosure describes embodiments of systems and methods for performing accuracy tests in the field or elsewhere to ensure that monitoring devices and their related systems are operating within acceptable tolerances. In some embodiments, a quality check insert has predetermined optical characteristics. When placed in a noninvasive sensor attached to patient monitor, the insert attenuates light from the sensor in a predetermined manner. Thus, the measurements made by the patient monitor can be compared to those expected to determine the accuracy of a given monitor.

In an embodiment, the monitoring devices measure a blood analyte, such as oxygen, carboxyhemoglobin, methemoglobin, total hemoglobin, proteins, glucose, lipids, a percentage thereof (e.g., saturation) or for measuring many other physiologically relevant patient characteristics. These characteristics can relate, for example, to pulse rate, hydration, trending information and analysis, and the like.

In some embodiments of a patient monitor, emitters in a sensor irradiate a quality check insert. The quality check insert attenuates, absorbs, and/or reflects the light from the emitters. The attenuated light passing through or reflected from the quality check insert is detected by detectors in the sensors. The patient monitor may numerically or graphically display various physiological parameters, including, but not limited to, a patient's plethysmograph (“pleth”),

In certain embodiments, a quality check insert may include water, other liquid(s), and/or other light absorbing constituents with known absorption and/or reflections characteristics. In an embodiment, those characteristics may match or substantially match expected norms and/or extremes of actual physiological parameters, or be entirely unrelated to physiological parameters. In some embodiments, the material composition itself of the quality check insert attenuates, absorbs, and/or reflects light in a predetermined manner that may or may not relate to norms or extremes of physiological parameters. Because the light absorbing constituents in the quality check insert are known, the expected value and/or range of values that a processor should provide after processing the detector signals will also be known. The known or predetermined parameter values are verification data, which can be a single value and/or a range of values. Values produced by the processing of the signals are field data. For quality control of a given patient monitor, the field data and verification data can be compared to determine if the patient monitor is functioning properly. When the quality check insert is properly positioned in the sensor, and the field data and verification data do not match, this can be an indication that the emitters, detectors, front end, cabling, and/or other parts of the patient monitor are not functioning properly. Conversely, if the field data and verification data match, this can be an indication that the system as whole is functioning properly.

In some embodiments, a quality control system can include the following: a noninvasive optical sensor configured to detect light attenuated by body tissue of a patient and output a detector signal responsive to the detected light; a patient monitor configured to process the detector signal to determine measurement values for one or more physiological parameters of the patient; and an insert shaped generally to mechanically mate with surfaces of the optical sensor, the insert having optical properties, wherein when the insert is properly placed within the sensor and irradiated by the sensor, the monitor processes detector signals, wherein the patient monitor provides display indicia indicative of whether the processed detector signals generate values within a predetermined range of values, the predetermined range associated with the optical properties of the insert.

In some embodiments, the quality control system can include one or more of the following: the insert has at least one of a color indicator or a symbol indicator corresponding to verification data; the verification data includes ranges of data; the optical properties of the insert are at least in part due to light absorbing constituents suspended within a body of the insert; further including an information element; the display indicia includes indicia indicating a quality pass or fail; the processing by the monitor includes processing detector signals when the insert is placed in the sensor is similar to processing detector signals when tissue is placed in the sensor; the processing by the monitor includes processing detector signals when the insert is placed in the sensor is different from processing detector signals when tissue is placed in the sensor; the patient monitor generates an audible indicia indicating a quality pass or fail; when the insert is properly placed within the sensor and irradiated by the sensor, the patient monitor determines whether an electric current draw of one or more light emitters of the sensor to generate a desired level of light intensity is within a predetermined current range of values; when the electric current draw is not within the predetermined current range of values, at least one of the light emitters of the sensor is determined to have failed; when the insert is properly placed within the sensor and irradiated by the sensor, the monitor processes the detector signals, wherein the patient monitor determines whether a gain level of the detector signal to generate a desired level of signal intensity is within a predetermined gain range of values; the detector signal is associated with one or more detectors of the sensor, and wherein when the gain level is not within the predetermined gain range of values, at least one of the detectors of the sensor is determined to have failed; when the insert is properly placed within the sensor, the patient monitor determines whether a rotation frequency of an active pulse motor of the sensor is within a predetermined frequency range of values; when the rotation frequency of the active pulse motor is not within the predetermined frequency range of values, the active pulse motor is determined to have failed; when the insert is properly placed within the sensor and attenuates light emitted by the sensor, the monitor processes the detector signals, and wherein the patient monitor determines whether a noise level associated with the detector signals is within a predetermined noise level range of values; the monitor processes the detector signals, and wherein the patient monitor determines whether a noise level associated with the detector signals is within a predetermined noise level range of values; when the insert is properly placed within the sensor, the patient monitor determines whether values generated by an acceleration signal associated with an accelerometer of the sensor are within a predetermined acceleration range of values; the patient monitor determines whether values generated by an acceleration signal associated with an accelerometer of the sensor are within a predetermined acceleration range of values; when the sensor is not moved and the values generated by the acceleration signal associated with the accelerometer are not within the predetermined acceleration range of values, the accelerometer is determined to have failed; when the insert is properly placed within the sensor, the patient monitor determines whether values generated by a temperature signal associated with a temperature sensor of the sensor are within a predetermined temperature range of values; the patient monitor determines whether values generated by a temperature signal associated with a temperature sensor of the sensor are within a predetermined temperature range of values; when ambient temperature is within an ambient temperature range corresponding to the predetermined temperature range of values and the values generated by the temperature signal associated with the temperatures sensor are not within the predetermined temperature range of values, the temperature sensor is determined to have failed; a body of the insert is sized to generally mechanically mate with the surfaces of the optical sensor that is a predetermined size, wherein the predetermined size of the optical sensor varies depending on a size of the body tissue of the patient desired to be inserted into the optical sensor; the insert has at least one of a color size indicator or a symbol size indicator corresponding to a predetermined size of the insert; the insert has a predetermined size, and each predetermined size of the body has a same predetermined range of values; and/or the insert has at least one of a color size indicator or a symbol size indicator corresponding to the predetermined size of the insert.

In some embodiments, a quality control system can include the following: a noninvasive optical sensor configured to detect light attenuated by body tissue of a patient and output a detector signal responsive to the detected light; a patient monitor configured to process the detector signal to determine measurement values for one or more physiological parameters of the patient; and an insert shaped generally to engage with surfaces of the optical sensor, the insert having optical properties, wherein when the insert is properly placed within the sensor and attenuates light emitted by the sensor, the monitor processes detector signals, wherein the patient monitor determines whether a processed detector signal generates a transmittance value within a predetermined transmittance range of values, the predetermined transmittance range associated with the optical properties of the insert.

In some embodiments, the quality control system can include one or more of the following: the patient monitor generates at least one of a visual indicia or an audible indicia indicating a quality pass or fail; when the insert is properly placed within the sensor and attenuates light emitted by the sensor, the patient monitor determines whether an electric current draw of one or more light emitters of the sensor to generate a desired level of light intensity is within a predetermined current range of values; when the electric current draw is not within the predetermined current range of values, at least one of the light emitters of the sensor is determined to have failed; when the insert is properly placed within the sensor and attenuates light emitted by the sensor, the monitor processes the detector signal, wherein the patient monitor determines whether a gain level of the detector signal to generate a desired level of signal intensity is within a predetermined gain range of values; the detector signal is associated with one or more detectors of the sensor, and wherein when the gain level is not within the predetermined gain range of values, at least one of the detectors of the sensor is determined to have failed; when the insert is properly placed within the sensor, the patient monitor determines whether a rotation frequency of an active pulse motor of the sensor is within a predetermined frequency range of values; when the rotation frequency of the active pulse motor is not within the predetermined frequency range of values, the active pulse motor is determined to have failed; when the insert is properly placed within the sensor and attenuates light emitted by the sensor, the monitor processes the detector signal, and wherein the patient monitor determines whether a noise level associated with the detector signals is within a predetermined noise level range of values; the monitor processes the detector signals, and wherein the patient monitor determines whether a noise level associated with the detector signals is within a predetermined noise level range of values; when the insert is properly placed within the sensor, the patient monitor determines whether values generated by an acceleration signal associated with an accelerometer of the sensor are within a predetermined acceleration range of values; the patient monitor determines whether values generated by an acceleration signal associated with an accelerometer of the sensor are within a predetermined acceleration range of values; when the sensor is not moved and the values generated by the acceleration signal associated with the accelerometer are not within the predetermined acceleration range of values, the accelerometer is determined to have failed; when the insert is properly placed within the sensor, the patient monitor determines whether values generated by a temperature signal associated with a temperature sensor of the sensor are within a predetermined temperature range of values; the patient monitor determines whether values generated by a temperature signal associated with a temperature sensor of the sensor are within a predetermined temperature range of values; when ambient temperature is within an ambient temperature range corresponding to the predetermined temperature range of values and the values generated by the temperature signal associated with the temperatures sensor are not within the predetermined temperature range of values, the temperature sensor is determined to have failed; the insert has at least one of a color indicator or a symbol indicator corresponding to verification data; the verification data includes ranges of data; the optical properties of the insert are at least in part due to light absorbing constituents in the insert; the light absorbing constituents are suspended within a body of the insert; the insert includes an information element corresponding to the optical properties, the information element configured to communicate the predetermined transmittance range of values to the monitor; the processing by the monitor includes processing detector signals when the insert is placed in the sensor is similar to processing detector signals when tissue is placed in the sensor; the processing by the monitor includes processing detector signals when the insert is placed in the sensor is different from processing detector signals when tissue is placed in the sensor; a body of the insert is sized to engage with the surfaces of the optical sensor that is a predetermined size, wherein the predetermined size of the optical sensor varies depending on a size of the body tissue of the patient desired to be inserted into the optical sensor; the insert has at least one of a color size indicator or a symbol size indicator corresponding to a predetermined size of the insert; the insert has a predetermined size, and each predetermined size of the body has a same predetermined transmittance range of values; and/or the insert has at least one of a color size indicator or a symbol size indicator corresponding to the predetermined size of the insert.

In some embodiments, a quality control insert for quality control testing of a noninvasive patient monitor can include the following: a body including light absorbing constituents having optical properties, the body configured to mate with a noninvasive optical sensor of a patient monitor configured to determine one or more physiological parameters of a patient, wherein the optical properties are associated with the light absorbing constituents attenuating light at predetermined light absorption values based on wavelengths of the light when the body of the insert is irradiated by the sensor.

In some embodiments, the quality control insert can include one or more of the following: the light absorbing constituents are suspended in the body of the insert; the body of the insert includes features configured to place the body in the sensor at a predetermined position and help retain the body in the sensor at the predetermined position; further include bumpers configured to aid in positioning the body in the sensor at a predetermined position; the bumpers include a stop configured to inhibit insertion of the body into the sensor beyond the predetermined position; the body includes an emitter outline for aligning the emitter outline with emitters of the sensor; the body includes an indentation that mirrors a bump of the sensor, the bump housing light detectors of sensor, the indentation mating with the bump when the body is inserted into the sensor at a predetermined position; the body includes a knob for holding the quality control insert during placement and alignment of the body in the sensor; further including at least one of a color indicator or a symbol indicator corresponding to the optical properties; further including an information element corresponding to the optical properties, the information element configured to communicate the predetermined absorption values to the patient monitor; the body is sized to be inserted into the optical sensor that is a predetermined size, wherein the predetermined size of the optical sensor varies depending on a size of body tissue of the patient desired to be inserted into the optical sensor; further including at least one of a color size indicator or a symbol size indicator corresponding to a predetermined size of the body; each predetermined size of the body has a same range of predetermined light absorption values; the body has a predetermined size, and each predetermined size of the body has a same range of predetermined light absorption values; the light absorbing constituents vary in at least one of type or quantity based on the predetermined size of the body; the light absorbing constituents vary in at least one of type or quantity based on a predetermined range of light absorption values; and/or further including at least one of a color indicator or a symbol indicator corresponding to the predetermined range of light absorption values, wherein the predetermined range of light absorption values corresponds to at least one of a high range or a low range of the light absorption values.

In some embodiments, a quality control method can include the following: inserting a quality control insert having optical properties into a noninvasive optical sensor of a patient monitor, the sensor configured to detect light attenuated by body tissue of a patient and output detector signals responsive to the detected light, the monitor configured to process the detector signals to determine measurement values for one or more physiological parameters of the patient; irradiating the insert in the sensor; detecting light attenuated by the insert; outputting a detector signal corresponding to the detected light attenuated by the insert; and processing the detector signal to determine whether the processed detector signal generates values within a predetermined range of values, the predetermined range of values associated with the optical properties of the insert.

In some embodiments, the quality control method can include one or more of the following: further including generating at least one of a visual indicia or an audible indicia indicating a quality pass or fail; the insert has at least one of a color indicator or a symbol indicator corresponding to verification data; the verification data includes ranges of data; the optical properties of the insert are at least in part due to light absorbing constituents in the insert; the light absorbing constituents are suspended within a body of the insert; further including reading an information element corresponding to the optical properties to communicate the predetermined range of values to the monitor; the processing by the monitor includes processing detector signals when the insert is placed in the sensor is similar to processing detector signals when tissue is placed in the sensor; the processing by the monitor includes processing detector signals when the insert is placed in the sensor is different from processing detector signals when tissue is placed in the sensor; further including determining whether an electric current draw of one or more light emitters of the sensor to generate a desired level of light intensity is within a predetermined current range of values; when the electric current draw is not within the predetermined current range of values, at least one of the light emitters of the sensor is determined to have failed; further including determining whether a gain level of the detector signals to generate a desired level of signal intensity is within a predetermined gain range of values; the detector signal is associated with one or more detectors of the sensor, and wherein when the gain level is not within the predetermined gain range of values, at least one of the detectors of the sensor is determined to have failed; further including determining whether a rotation frequency of an active pulse motor of the sensor is within a predetermined frequency range of values; when the rotation frequency of the active pulse motor is not within the predetermined frequency range of values, the active pulse motor is determined to have failed; further including determining whether a noise level associated with the detector signals is within a predetermined noise level range of values; further including determining whether values generated by an acceleration signal associated with an accelerometer of the sensor are within a predetermined acceleration range of values; when the sensor is not moved and the values generated by the acceleration signal associated with the accelerometer are not within the predetermined acceleration range of values, the accelerometer is determined to have failed; further including determining whether values generated by a temperature signal associated with a temperature sensor of the sensor are within a predetermined temperature range of values; when ambient temperature is within an ambient temperature range corresponding to the predetermined temperature range of values and the values generated by the temperature signal associated with the temperatures sensor are not within the predetermined temperature range of values, the temperature sensor is determined to have failed; further including choosing a size of the quality control insert to insert into the optical sensor that is a predetermined size, wherein the predetermined size of the optical sensor varies depending on a size of the body tissue of the patient desired to be inserted into the optical sensor; the choosing of the size of the quality control insert is based on at least one of a color size indicator or a symbol size indicator corresponding to a predetermined size of the insert corresponding to the predetermined size of the optical sensor; each size of the insert has a same predetermined transmittance range of values; and/or the insert has at least one of a color size indicator or a symbol size indicator corresponding to the size of the insert.

In some embodiments, a quality control kit can include the following: a plurality of quality control inserts, each insert including light absorbing constituents having predetermined optical properties, each insert configured to mate with a noninvasive optical sensor of a patient monitor configured to determine one or more physiological parameters of a patient, wherein the predetermined optical properties are associated with the light absorbing constituents attenuating light at predetermined light absorption values based on wavelengths of the light when the insert is irradiated by the sensor.

In some embodiments, the quality control kit can include one or more of the following: each insert is sized to be inserted into a predetermined size sensor, wherein the predetermined size sensor varies depending on a size of body tissue of the patient desired to be inserted into the sensor; each insert has at least one of a color size indicator or a symbol size indicator corresponding to a predetermined size of the insert; the light absorbing constituents of each insert vary in at least one of type or quantity based on a predetermined range of light absorption values; each insert has at least one of a color indicator or a symbol indicator corresponding to the predetermined range of light absorption values, wherein the predetermined range of light absorption values corresponds to at least one of a high range or a low range of the light absorption values; and/or each insert has a same predetermined range of light absorption values.

For purposes of summarizing the disclosure, certain aspects, advantages and novel features of the disclosures have been described herein. It is to be understood that not necessarily all such advantages can be achieved in accordance with any particular embodiment of the disclosures disclosed herein. Thus, the disclosures disclosed herein can be embodied or carried out in a manner that achieves or optimizes one advantage or group of advantages as taught herein without necessarily achieving other advantages as can be taught or suggested herein.

### BRIEF DESCRIPTION OF THE DRAWINGS

FIG. 1 illustrates a simplified exemplary perspective view of a kit including a plurality of quality check inserts according to an embodiment of this disclosure.

FIG. 2 illustrates a simplified exemplary embodiment of the quality check insert and a simplified exemplary perspective view of a processing system according to an embodiment of present disclosure, including a processing device, a noninvasive sensor, and a cable providing communication between the device and the sensor.

FIG. 3 illustrates a simplified exemplary embodiment of the quality check insert and a simplified exemplary perspective view of a processing system according to another embodiment of present disclosure, including a processing device, a noninvasive sensor, and a cable providing communication between the device and the sensor.

FIG. 4 illustrates a simplified exemplary embodiment of the quality check insert and a simplified exemplary perspective view of a processing system according to another embodiment of present disclosure, including a processing device, a noninvasive sensor, and a cable providing communication between the device and the sensor.

FIG. 5 illustrates a simplified exemplary embodiment of the quality check insert and a simplified exemplary side view of a noninvasive optical sensor.

FIG. 6 illustrates a simplified exemplary embodiment of the quality check insert and a simplified exemplary perspective view of a noninvasive optical sensor.

FIG. 7 illustrates a simplified exemplary embodiment of the quality check insert and a simplified exemplary block diagram of a physiological monitor.

FIG. 8 is a simplified exemplary graph of absorbance versus wavelength curve exhibited by, for example, a quality check insert according to an embodiment of this disclosure.

FIG. 9 illustrates a simplified exemplary embodiment of the quality check insert and a simplified exemplary perspective view of a processing system according to an embodiment of present disclosure, including a processing device displaying verification data.

FIGS. 10A, 10B, and 100 illustrate simplified exemplary embodiments of a quality check insert.

FIG. 11A illustrates a top-front perspective view of a simplified exemplary embodiment of a quality check insert.

FIG. 11B illustrates a top-back perspective view of a simplified exemplary embodiment of a quality check insert.

FIG. 110 illustrates bottom-front perspective view of a simplified exemplary embodiment of a quality check insert.

FIG. 11D illustrates a bottom-back perspective view of a simplified exemplary embodiment of the quality check insert.

FIG. 12 illustrates a cutaway view of a simplified exemplary embodiment of a noninvasive optical sensor.

FIG. 13 is a block diagram of a simplified exemplary embodiment of a quality control system for quality control testing.

### DETAILED DESCRIPTION

In this application, reference is made to many blood parameters. Some references that have common shorthand designations are referenced through such shorthand designations. For example, as used herein, HbCO designates carboxyhemoglobin, HbMet designates methemoglobin, and Hbt designates total hemoglobin. Other shorthand designations such as COHb, MetHb, and tHb are also common in the art for these same constituents. These constituents are generally reported in terms of a percentage, often referred to as saturation, relative concentration or fractional saturation. Total hemoglobin is generally reported as a concentration in g/dL. The use of the particular shorthand designators presented in this application does not restrict the term to any particular manner in which the designated constituent is reported.

FIG. 1 illustrates embodiments of quality check inserts 102 having known optical characteristics. For example, the insert 102 may absorb and/or reflect light similar to or the same tissue. The quality check insert 102 may advantageously include absorption characteristics the same as or similar to tissue having physiological parameter values within clinical data norms or data outliers. The term “parameters” can refer to any of the aforementioned types of parameters.

The quality check inserts 102 are discussed in further detail with reference to FIGS. 10A-B. In some embodiments, the quality check insert 102 is generally shaped like a cylinder, or more specifically, generally shaped like a patient's finger. In other embodiments, the quality check insert can be shaped like a patient's toe, earlobe, or the like.

### Patient Monitors

FIG. 2 illustrates an example of a processing system 200. In the depicted embodiment, the processing system 200 includes a processing device, patient monitor, or instrument 209, a finger clip sensor 201 connected to the monitor 209 via a cable 212. The finger clip sensor can be adapted to removably attach to, and transmit light through, a fingertip or a quality check insert 102. The sensor cable 212 and monitor connector 211 are integral to the sensor 201, as shown. In alternative embodiments, the sensor 201 may be configured separately from the cable 212 and connector 211. In the embodiment shown, the monitor 209 includes a display 210, control buttons 208 and a power button 214. Moreover, the monitor 209 can advantageously include various electronic processing, signal processing, and data storage devices capable of receiving signal data from said sensor 201, processing the signal data to determine one or more output measurement values indicative of one or more physiological parameters of a monitored patient or non-physiological parameters. In the present embodiment, the same or similar processing may be applied to one or more quality check inserts 102. In various embodiments, the monitor 209 advantageously displays the measurement values (whether they correspond to physiological measurements or to the quality check inserts 102, trends of the measurement values, combinations of measurement values, and the like.

The cable 212 connecting the sensor 201 and the monitor 209 can be implemented using one or more wires, optical fiber, flex circuits, or the like. In some embodiments, the cable 212 can employ twisted pairs of conductors in order to minimize or reduce cross-talk of data transmitted from the sensor 201 to the monitor 209. Various lengths of the cable 212 can be employed to allow for separation between the sensor 201 and the monitor 209. The cable 212 can be fitted with a connector (male or female) on either end of the cable 212 so that the sensor 201 and the monitor 209 can be connected and disconnected from each other. Alternatively, the sensor 201 and the monitor 209 can be coupled together via a wireless communication link, such as an infrared link, radio frequency channel, or any other wireless communication protocol and channel. A wireless communication link system is described in more detail in U.S. Pat. No. 6,850,788, incorporated by reference herein.

The monitor 209 can be attached to the patient. For example, the monitor 209 can include a belt clip or straps that facilitate attachment to a patient's belt, arm, leg, or the like. The monitor 209 can also include a fitting, slot, magnet, LEMO snap-click connector, or other connecting mechanism to allow the cable 212 and sensor 201 to be attached to the monitor 209.

The monitor 209 can also include other components, such as a speaker, removable storage and/or memory (e.g., a flash card slot), an AC power port, and one or more network interfaces, such as a universal serial bus interface or an Ethernet port. For example, the monitor 209 can include a display 210 that can indicate a measurement of tHb, such as, for example, “SpHb,” or other parameters such as SpO₂, pulse rate, and/or perfusion index. Other analytes and forms of display can also appear on the monitor 209. In an embodiment, the monitor 209 includes an integral or detachable glucose strip reader. A detachable glucose strip reader can be separately housed and configured to communicate wirelessly with monitor 209 or by attachment to a network interface, universal serial bus interface or Ethernet port. In an embodiment, an invasive glucose strip test device can be integrated into the monitor 209 or as a separate dongle connectable, for example, through the sensor cable port, or the like. The invasive glucose strip test can be used to calibrate a non-invasive optical glucose measurement. The strip test device can be used as a measure for measurements not performed by the monitor 209 or in addition to other measurements performed by the monitor 209. In an embodiment, blood pressure measurements can also be integrated into the monitor 209.

Although a single sensor 201 with a single monitor 209 is shown, different combinations of sensors and device pairings can be implemented. For example, multiple sensors can be provided for a plurality of differing patient types or measurement sites or even patient fingers. In an embodiment, a resposable sensor can be used. A resposable sensor integrates both reusable and disposable components. For example, the emitters, detectors and motor assembly can be reused while the components used to attach the sensor to the patient can be disposable. An active pulse system is described in more detail in U.S. patent application Ser. No. 13/473,477 titled “Personal Health Device,” filed on May 16, 2012, the disclosure of which is hereby incorporated by reference in its entirety.

FIG. 3 illustrates a simplified perspective view of another embodiment of a monitoring or processing system 300, including a processing device, patient monitor, or instrument 302, a noninvasive sensor 304, an associated cable 306 providing communication between the device 302 and the sensor 304. The processing device 302 comprises a handheld housing including an integrated touch screen 310, one or more input keys 312, and an integrated camera 313 preferably capable of photo and/or video capture. In an embodiment, the screen 310 rotates as the device 302 is held in differing orientations; however, the preferred orientation is for use is the landscape.

FIG. 3 also illustrates additional features of the device 302. For example, the device 302 may include along a side thereof an integrated strip reader, including a strip input cavity 314, and a power button 316. Along another side, the device 302 includes a noninvasive sensor cable input port 320 and volume controls 322 (detail not visible from the perspective view of FIG. 3). Along yet another side, the device 302 includes a headphone jack 324, a micro SD card reader input cavity 326, a micro HDMI connector 328, a Micro USB connector 330 configured for, for example, data transfer and battery charging, and an optional audio transducer, such as, for example, a speaker 332. Along a back side thereof, in an embodiment, the processing device 302 includes a camera and LED flash 136 (detail not visible from the perspective view of FIG. 3).

As disclosed, the device 302 communicates with a noninvasive optical sensor 304, such as, for example, a clothespin style reusable optical sensor, in some mechanical respects similar to those employed in standard pulse oximetry. The sensor 304 may also include advanced features, such as those disclosed in U.S. Pat. No. 6,580,086, and U.S. Pat. Pub. No. 2010-0026995, on Feb. 4, 2010, titled “Multi-stream Sensor For Noninvasive Measurement of Blood Constituents,” each disclosure of which is hereby incorporated by reference in their entirety. Specifically, the sensor 304 includes a plurality of emitters emitting light of a variety of wavelengths to form a light source to irradiate or impinge light on a patient tissue. A plurality of detectors detect the light after attenuation by a digit of the patient or quality check insert 102. A plurality of temperature sensors and one or more memory devices may also be incorporated into the sensor 304. These devices communicate their information to the device 302 through the cable 306.

In general, the user interacts with the processing device 302 to obtain glucose measurements. The user may input a disposable strip with a blood sample and the device 302 will, if not already, electronically wake up a medical application and display glucose measurements obtained from the strip reader. The user may also apply the sensor 304 to a digit or quality check insert 102 and upon activating a “test” input, the device 302 may process the detector signals and display glucose or other measurements derived from the received signals.

Although disclosed with respect to the embodiment shown in FIG. 3, an artisan will recognize from the disclosure herein alternative or additional functionality, user interaction mechanisms, and the like. For example, the device housing may be shaped to ergonomically fit a user's hand, may include more or less input mechanisms including, for example, a connectable or slideout keyboard, a pointing device, speech recognition applications, or the like. Moreover, the sensor 304 may wirelessly communicate with the device 302. The device 302 may communicate with an external strip reader or other medical sensors or devices. A processing system is described in more detail in 7,764,982, and in U.S. patent application Ser. No. 13/651,167, titled “Medical Monitoring Hub,” filed Oct. 12, 2012, the disclosures of which are hereby incorporated by reference in its entirety.

FIG. 4 illustrates another embodiment of a processing system 400 having a processing device, patient monitor, or instrument 402 and a multiple wavelength sensor assembly 404 with enhanced measurement capabilities as compared with conventional pulse oximetry. The physiological processing system 400 allows the monitoring of a person, including a patient, or quality check insert 102. In particular, the multiple wavelength sensor assembly 404 allows the measurement of blood constituent and related parameters in addition to oxygen saturation and pulse rate. Alternatively, the multiple wavelength sensor assembly 404 allows the measurement of oxygen saturation and pulse rate with increased accuracy or robustness as compared with conventional pulse oximetry. The processing system 400 can advantageously include various electronic processing, signal processing, and data storage devices capable of receiving signal data from the sensor assembly 404, processing the signal data to determine one or more output measurement values indicative of one or more physiological parameters of a monitored patient or non-physiological parameters for some embodiments of the quality check insert 102, and displaying the measurement values, trends of the measurement values, combinations of measurement values, and the like.

In one embodiment, the sensor assembly 404 is configured to plug into a monitor sensor port 410. Monitor keys 460 provide control over operating modes and alarms, to name a few. A display 470 provides readouts of measured parameters, such as oxygen saturation, pulse rate, HbCO and HbMet to name a few. A patient monitor is described in more detail in 7,764,982, and in U.S. patent application Ser. No. 13/651,167, titled “Medical Monitoring Hub,” filed Oct. 12, 2012, the disclosures of which are hereby incorporated by reference in its entirety.

Referring to FIG. 5, the sensor 501 in the depicted embodiment is a clothespin-shaped clip sensor that includes an enclosure 502 for receiving a patient's finger. The enclosure 502 is formed by an upper section or emitter shell 504, which is rotatably or pivotally connected with a lower section or detector shell 506. The emitter shell 504 can be biased with the detector shell 506 to close together around a pivot point 503 and thereby sandwich finger tissue or a quality check insert 102 between the emitter and detector shells 504, 506.

In an embodiment, the pivot point 503 advantageously includes a pivot capable of adjusting the relationship between the emitter and detector shells 504, 506 to effectively level the sections when applied to a tissue site or quality check insert 102. In another embodiment, the sensor 501 includes some or all features of the finger clip described in U.S. Pat. No. 7,764,982, incorporated above, such as a spring that causes finger clip forces to be distributed along the finger. Cols. 13-15, which describe this feature, are hereby specifically incorporated by reference. Other pivot points as disclosed in other incorporated patent filings referenced above also provide disclosure of springs and their effect on sensor mechanisms to distribute forces over the finger.

The emitter shell 504 can position and house various emitter components of the sensor 501. It can be constructed of reflective material (e.g., white silicone or plastic) and/or can be metallic or include metalized plastic (e.g., including carbon and aluminum) to possibly serve as a heat sink. The emitter shell 504 can also include absorbing opaque material, such as, for example, black or grey colored material, at various areas, such as on one or more flaps 507, to reduce ambient light entering the sensor 501.

The detector shell 506 can position and house one or more detector portions of the sensor 501. The detector shell 506 can be constructed of reflective material, such as white silicone or plastic. As noted, such materials can increase the usable signal at a detector by forcing light back into the tissue and measurement site (see FIG. 1). The detector shell 506 can also include absorbing opaque material at various areas, such as lower area 508, to reduce ambient light entering the sensor 501.

FIG. 6 illustrates another view of the sensor 501, which includes an embodiment of a partially cylindrical protrusion 605. The finger bed 610 includes a generally curved surface shaped generally to receive tissue, such as a human digit. The finger bed 610 also includes the ridges or channels 614. The finger bed 610 can include other one or more protrusions, bumps, lenses, or other suitable mechanisms for shaping tissue or a quality check insert. The finger bed 610 shown also includes the protrusion 605. The protrusion 605, ridges or channels 614, and/or other suitable mechanisms can help prevent twisting of a quality check insert once positioned inside the sensor. In one embodiment, the quality check insert 602 has contours or features 603 that are shaped to mate with the finger bed 610, particularly, the protrusion 605 and/or ridges or channels 614 as discussed in further detail herein, although approximate or identical mechanical mating may assist in the initial and subsequent alignment of the quality check insert 610 within the sensor cavity. The mating of the quality check insert 602 with the sensor 501 can help ensure that the quality check insert 602 is properly positioned for the measurement of physiological and/or non-physiological parameters. Further, the mating can help ensure that the quality check insert 602 does not shift or get dislodged once positioned inside the sensor 501.

Patient Monitors with Active Pulse

A typical heart beats around 1 Hz creating a fairly predictable heart rate. Determining the heart rate is important for many applications and particularly important for pulse oximetry and noninvasive determination of other parameters using pulse oximetry techniques. This is because the pulse affects light absorption rates at predictable amounts. Thus, knowing the pulse rate is essential to determining accurate non-invasive optical measurements. This information is useful for determining various physiological parameters. These parameters include, for example, a percent value for HbCO (“SpCO”), a percent value for HbMet (“SpMet”), fractional SpO₂ (“FpO₂”) or the like. Additionally, caregivers often desire knowledge of blood glucose, total hematocrit (Hct), bilirubin, pulse rate, perfusion quality, signal quality or the like.

Similarly, introducing an artificial excitation can cause perturbations in the blood flow similar to the effects of a heartbeat. These artificial excitations can be used as an alternative to the natural pulse rate or in addition to the natural pulse rate. Artificial excitations also advantageously are excited at known frequencies. Thus, it is not necessary to first determine the pulse rate of an individual. However, a frequency of such artificial excitations should not overlap with a frequency of the heart rate or its harmonics. In one embodiment, an excitation frequency of five to six times the natural heart rate can be chosen. Moreover, it is also important to provide artificial excitations at frequencies that do not cause discomfort to the patient. Thus, a range of frequencies that are useful for artificial excitations includes a range of about 6 Hz to about 30 Hz. An active pulse system is described in more detail in U.S. Publication No. 2012/0296178 titled “Personal Health Device,” filed on May 16, 2012, the disclosure of which is hereby incorporated by reference in its entirety.

In an embodiment, a quality check insert 102 can have channels or veins having known absorption/reflection characteristics as will be discussed in further detail with reference to FIG. 100. The quality check insert 102 can cause the field data to be different from the verification data if the artificial excitation is not functioning properly even though the emitters and detectors may be functioning properly. The active pulse system can be activated to cause artificial excitations in the quality check insert 102. In other embodiments of FIGS. 10A-B, the active pulse system can be activated to cause artificial excitations in the quality check insert 102 without affecting the field data.

FIG. 7 illustrates an example of a data collection system 700 with an active pulse feature. In another embodiment, the same data collection system except without the active pulse feature can be used for quality control with embodiments of the quality check insert having known absorption/reflection characteristics that do not require artificial activation. In certain embodiments, the data collection system 700 noninvasively measures a blood analyte, such as oxygen, carbon monoxide, methemoglobin, total hemoglobin, proteins, glucose, lipids, a percentage thereof (e.g., saturation) or for measuring many other physiologically relevant patient characteristics, or non-physiological parameters for some embodiments of the quality check insert. The system 700 can also measure additional blood analytes and/or other physiological parameters useful in determining a state or trend of wellness of a patient.

The data collection system 700 can measure optical radiation from the measurement site such as a digit or a quality check insert. The optical radiation can be used to determine analyte concentrations, including glucose, total hemoglobin, methemoglobin, carboxyhemoglobin, oxygen saturation, etc., at least in part by detecting light attenuated by a measurement site 702. The measurement site 702 can be any location on a patient's body, such as a finger, foot, ear lobe, or the like or a quality check insert. This disclosure is described primarily in the context of a quality check insert measurement site 702. However, the features of the embodiments disclosed herein can be used with other measurement sites 702.

In the depicted embodiment, the system 700 includes an optional tissue thickness adjuster or tissue/insert shaper 705, which can include one or more protrusions, bumps, lenses, or other suitable tissue-shaping mechanisms. In certain embodiments, the tissue/insert shaper 705 is a flat or substantially flat surface that can be positioned proximate the measurement site 702 and that can apply sufficient pressure to cause the tissue or quality check insert of the measurement site 702 to be flat or substantially flat. In other embodiments, the tissue/insert shaper 705 is a convex or substantially convex surface with respect to the measurement site 702. Many other configurations of the tissue/insert shaper 705 are possible. Advantageously, in certain embodiments, the tissue/insert shaper 705 reduces thickness of the measurement site 702 while preventing or reducing occlusion at the measurement site 702. Reducing thickness of the site can advantageously reduce the amount of attenuation of the light because there is less tissue or quality check insert through which the light must travel. Shaping the tissue or quality check insert into a convex (or alternatively concave) surface can also provide more surface area from which light can be detected.

The embodiment of the data collection system 700 shown also includes an optional noise shield 703. In an embodiment, the noise shield 703 can be advantageously adapted to reduce electromagnetic noise while increasing the transmittance of light from the measurement site 702 to one or more detectors 706 (described below). For example, the noise shield 703 can advantageously include a conductive coated glass or metal grid electrically communicating with one or more other shields of the sensor 701 or electrically grounded. Also included is an active pulse motor 720 (described below).

The data collection system 700 can include a sensor 701 (or multiple sensors) that is coupled to a processing device or monitor 709. In an embodiment, the sensor 701 and the monitor 709 are integrated together into a single unit. In another embodiment, the sensor 701 and the monitor 709 are separate from each other and communicate one with another in any suitable manner, such as via a wired or wireless connection. The sensor 701 and monitor 709 can be attachable and detachable from each other for the convenience of the user or caregiver, for ease of storage, sterility issues, or the like. The sensor 701 and the monitor 709 will now be further described.

In the depicted embodiment shown in FIG. 7, the sensor 701 includes an emitter 704, a tissue/insert shaper 705, a set of detectors 706, and a front-end interface 708. The emitter 704 can serve as the source of optical radiation transmitted towards measurement site 702. As will be described in further detail below, the emitter 704 can include one or more sources of optical radiation, such as LEDs, laser diodes, incandescent bulbs with appropriate frequency-selective filters, combinations of the same, or the like. In an embodiment, the emitter 704 includes sets of optical sources that are capable of emitting visible and near-infrared optical radiation.

In some embodiments, the emitter 704 is used as a point optical source, and thus, the one or more optical sources of the emitter 704 can be located within a close distance to each other, such as within about a 2 mm to about 4 mm. The emitters 704 can be arranged in an array, such as is described in U.S. Publication No. 2006/0211924, filed Sep. 21, 2006, titled “Multiple Wavelength Sensor Emitters,” the disclosure of which is hereby incorporated by reference in its entirety. In particular, the emitters 704 can be arranged at least in part as described in paragraphs [0061] through [0068] of the aforementioned publication, which paragraphs are hereby incorporated specifically by reference. Other relative spatial relationships can be used to arrange the emitters 704.

The data collection system 700 also includes a driver 711 that drives the emitter 704. The driver 711 can be a circuit or the like that is controlled by the monitor 709. For example, the driver 711 can provide pulses of current to the emitter 704. In an embodiment, the driver 711 drives the emitter 704 in a progressive fashion, such as in an alternating manner. The driver 711 can drive the emitter 704 with a series of pulses of about 1 milliwatt (mW) for some wavelengths that can penetrate tissue relatively well and from about 40 mW to about 100 mW for other wavelengths that tend to be significantly absorbed in tissue. A wide variety of other driving powers and driving methodologies can be used in various embodiments.

The driver 711 can be synchronized with other parts of the sensor 701 and can minimize or reduce jitter in the timing of pulses of optical radiation emitted from the emitter 704. For example, in an embodiment, the timing of the pulses is synchronized with the timing of the motor 720 revolutions. In some embodiments, the driver 711 is capable of driving the emitter 704 to emit optical radiation in a pattern that varies by less than about 10 parts-per-million.

The detectors 706 capture and measure light from the measurement site 702. For example, the detectors 706 can capture and measure light transmitted from the emitter 704 that has been attenuated or reflected from the tissue or quality check insert in the measurement site 702. The detectors 706 can output a detector signal 707 responsive to the light captured or measured. The detectors 706 can be implemented using one or more photodiodes, phototransistors, or the like.

In addition, the detectors 706 can be arranged with a spatial configuration to provide a variation of path lengths among at least some of the detectors 706. That is, some of the detectors 706 can have the substantially, or from the perspective of the processing algorithm, effectively, the same path length from the emitter 704. However, according to an embodiment, at least some of the detectors 706 can have a different path length from the emitter 704 relative to other of the detectors 706. Variations in path lengths can be helpful in allowing the use of a bulk signal stream from the detectors 706. In some embodiments, the detectors 706 may employ a linear spacing, a logarithmic spacing, or a two or three dimensional matrix of spacing, or any other spacing scheme in order to provide an appropriate variation in path lengths.

Active Pulse Motor 720 rotates providing an agitation at a known frequency which is transferred through the sensor to the measurement site. The motor 720 is driven by driver 711. The vibration created by the motor 720 is useful in determining further information regarding the physiological state of the patient as described in more detail in U.S. patent application Ser. No. 13/473,477 titled “Personal Health Device,” filed on May 16, 2012, the disclosure of which is hereby incorporated by reference in its entirety.

The front end interface 708 provides an interface that adapts the output of the detectors 706, which is responsive to desired physiological and/or non-physiological parameters for some embodiments of the quality check insert. For example, the front end interface 708 can adapt a signal 707 received from one or more of the detectors 706 into a form that can be processed by the monitor 709, for example, by a signal processor 710 in the monitor 709. The front end interface 708 can have its components assembled in the sensor 701, in the monitor 709, in connecting cabling (if used), combinations of the same, or the like. The location of the front end interface 708 can be chosen based on various factors including space desired for components, desired noise reductions or limits, desired heat reductions or limits, and the like.

The front end interface 708 can be coupled to the detectors 706 and to the signal processor 710 using a bus, wire, electrical or optical cable, flex circuit, or some other form of signal connection. The front end interface 708 can also be at least partially integrated with various components, such as the detectors 706. For example, the front end interface 708 can include one or more integrated circuits that are on the same circuit board as the detectors 706. Other configurations can also be used.

The front end interface 708 can be implemented using one or more amplifiers, such as transimpedance amplifiers, that are coupled to one or more analog to digital converters (ADCs) (which can be in the monitor 709), such as a sigma-delta ADC. A transimpedance-based front end interface 708 can employ single-ended circuitry, differential circuitry, and/or a hybrid configuration. A transimpedance-based front end interface 708 can be useful for its sampling rate capability and freedom in modulation/demodulation algorithms. For example, this type of front end interface 708 can advantageously facilitate the sampling of the ADCs being synchronized with the pulses emitted from the emitter 704 and/or vibrations from the motor 720.

The ADC or ADCs can provide one or more outputs into multiple channels of digital information for processing by the signal processor 710 of the monitor 709. Each channel can correspond to a signal output from a detector 706.

In some embodiments, a programmable gain amplifier (PGA) can be used in combination with a transimpedance-based front end interface 708. For example, the output of a transimpedance-based front end interface 708 can be output to a PGA that is coupled with an ADC in the monitor 709. A PGA can be useful in order to provide another level of amplification and control of the stream of signals from the detectors 706. Alternatively, the PGA and ADC components can be integrated with the transimpedance-based front end interface 708 in the sensor 701.

In another embodiment, the front end interface 708 can be implemented using switched-capacitor circuits. A switched-capacitor-based front end interface 708 can be useful for, in certain embodiments, its resistor-free design and analog averaging properties. In addition, a switched-capacitor-based front end interface 708 can be useful because it can provide a digital signal to the signal processor 710 in the monitor 709.

As shown in FIG. 7, the monitor 709 can include the signal processor 710 and a user interface, such as a display 712. The monitor 709 can also include optional outputs alone or in combination with the display 712, such as a storage device 714 and a network interface 716. In an embodiment, the signal processor 710 includes processing logic that determines measurements for desired analytes, such as glucose and total hemoglobin, based on the signals received from the detectors 706. The signal processor 710 can be implemented using one or more microprocessors or subprocessors (e.g., cores), digital signal processors, application specific integrated circuits (ASICs), field programmable gate arrays (FPGAs), combinations of the same, and the like.

The signal processor 710 can provide various signals that control the operation of the sensor 701. For example, the signal processor 710 can provide an emitter control signal to the driver 711. This control signal can be useful in order to synchronize, minimize, or reduce jitter in the timing of pulses emitted from the emitter 704 or motor vibrations from motor 720. Accordingly, this control signal can be useful in order to cause optical radiation pulses emitted from the emitter 704 to follow a precise timing and consistent pattern. For example, when a transimpedance-based front end interface 708 is used, the control signal from the signal processor 710 can provide synchronization with the ADC in order to avoid aliasing, cross-talk, and the like. As also shown, an optional memory 713 can be included in the front-end interface 708 and/or in the signal processor 710. This memory 713 can serve as a buffer or storage location for the front-end interface 708 and/or the signal processor 710, among other uses.

The user interface 712 can provide an output, e.g., on a display, for presentation to a user of the data collection system 700. The user interface 712 can be implemented as a touch-screen display, an LCD display, an organic LED display, or the like. In addition, the user interface 712 can be manipulated to allow for measurement on the non-dominant side of the patient. For example, the user interface 712 can include a flip screen, a screen that can be moved from one side to another on the monitor 709, or can include an ability to reorient its display indicia responsive to user input or device orientation. In alternative embodiments, the data collection system 700 can be provided without a user interface 712 and can simply provide an output signal to a separate display or system.

A storage device 714 and a network interface 716 represent other optional output connections that can be included in the monitor 709. The storage device 714 can include any computer-readable medium, such as a memory device, hard disk storage, EEPROM, flash drive, or the like. The various software and/or firmware applications can be stored in the storage device 714, which can be executed by the signal processor 710 or another processor of the monitor 709. The storage device 714 can include verification data 715, which is compared to field data as described in further detail herein. The network interface 716 can be a serial bus port (RS-232/RS-485), a Universal Serial Bus (USB) port, an Ethernet port, a wireless interface (e.g., WiFi such as any 802.1x interface, including an internal wireless card), or other suitable communication device(s) that allows the monitor 709 to communicate and share data with other devices. The monitor 709 can also include various other components not shown, such as a microprocessor, graphics processor, or controller to output the user interface 712, to control data communications, to compute data trending, or to perform other operations. In an embodiment, the measurements are encrypted and decrypted inside the processor in hardware. As a result, the measurements can be safely stored and communicated to, for example, a cloud based storage medium without compromising the security of the data.

Although not shown in the depicted embodiment, the data collection system 700 can include various other components or can be configured in different ways. For example, the sensor 701 can have both the emitter 704 and detectors 706 on the same side of the measurement site 702 and use reflectance to measure analytes. The data collection system 700 can also include a sensor that measures the power of light emitted from the emitter 704.

### Quality Check Insert

FIG. 8 is an example of absorption μₐ, 820 versus wavelength 830 characteristics of an embodiment of a quality check insert. The graph of FIG. 8 illustrates a quality control absorbance profile 810. The quality check insert can have light attenuation characteristics of the absorbance profile 810 when a patient monitor irradiates the quality check insert 102 as discussed herein. Table 1 below shows example values of wavelength and absorbance corresponding to the absorbance profile 810. In some embodiments, Table 1 can be a look up table that is stored in the patient monitor for use during quality control. The look up table can have values that are more or less precise, including ranges as discussed herein. During quality control, the patient monitor may irradiate the quality check insert, detect the attenuated light, and process the detected attenuated light signals to determine measurement values associated with the insert. These values can then be compared to known values associated with insert.

TABLE 1

| Wavelength | Absorbance |
|---|---|
| 660.1133 | 0.955812 |
| 915.9829 | 0.298169 |
| 964.1929 | 0.191983 |
| 1043.723 | 0.143996 |
| 1191.04 | 0.440637 |
| 1267.781 | 0.258849 |
| 1286.124 | 0.250979 |
| 1310.986 | 0.292902 |

Continuing with the example of Table 1, in an embodiment, while irradiating the quality check insert, the patient monitor may advantageously compute a number of normalized ratios or ratio data. The insert has a known absorbance of about 0.96 at a wavelength of about 660 nanometers (nm), an absorbance of about 0.14 at a wavelength of about 1043 nm, and an absorbance of about 0.44 at a wavelength of about 1191 nm, again with the foregoing values being indicative of normalized ratio data. When the detected absorbance values correspond to the absorbance values of the absorbance profile 810 and/or Table 1, or a range thereof, the patient monitor can be deemed to have passed quality control and to be functioning properly. A quality control pass or fail indication can be communicated to a user, for example, on a screen or display, or through an auditory signal.

In some embodiments, the patient monitor irradiates the quality check insert while rotating the active pulse motor. During quality control, the patient monitor can monitor the frequency of rotation (output frequency of the motor) and determine if the output frequency is within a predetermined range of rotation frequencies corresponding to useful artificial excitation frequencies as discussed herein.

During quality control, light emitters of an optional sensor can irradiate the quality check insert. The quality control can attenuate light as discussed herein. Detectors of the optical sensor can generate signal data indicative of the attenuated light. The patient monitor can process the signal data to, for example, obtain normalized ratios or ratio data corresponding to the light attenuated by the quality check insert. In an embodiment, active pulse motor rotation can cause perturbations in the quality check insert material as discussed herein. The detector generated signal data can correspond to alternating current (AC) signal data indicative of the perturbations in the quality check insert material and light transmittance of the quality check insert. The patient monitor can process the AC signal data at least partly based on one or more quality control algorithms (e.g., as discussed herein, and in particular, in reference to FIG. 13) to derive intensity of the transmitted light at least party based on intensity of the light emitters, PGA (gain of the system), power of the light emitters, and/or light transmittance of the quality check insert. Using one or more quality control algorithms, the patient monitor can determine the intensity of attenuated light corresponding to absorbance values or range of values of the quality check insert as discussed in reference to FIG. 8 and Table 1, as well as determine output rotation frequency of the active pulse motor based on frequency modulation of the AC signal data corresponding to the normalized ratios or ratio data.

In some embodiments, quality control can be performed without rotation or excitation by an active pulse motor. The detector generated signal data can correspond to direct current (DC) signal data indicative of light transmittance of the quality check insert. The patient monitor can process the DC signal data at least partly based on one or more quality control algorithms (as discussed herein, and in particular, in reference to steps 1310, 1312, 1314, 1316, and 1318 of FIG. 13). In some embodiments, the patient monitor can process the DC signal data at least partly based on other algorithms. The patient monitor can determine the intensity of attenuated light corresponding to absorbance values or range of values similarly to processing AC signal data as discussed herein.

Referring to FIG. 1, the quality check inserts 102 can have a body 104, 106, 108 having known absorption/reflection characteristics. In one embodiment, the characteristics may be similar to or the same as tissue having high values of, for example, tHb and/or SpO₂. In another embodiment, the characteristics may be similar to or the same as tissue having generally medium values of, for example, tHb or SpO₂. In yet another embodiment, the characteristics may be similar to or the same as tissue having generally low values of, for example, tHb and SpO₂. The insert may exhibit optical properties that are the same or similar to tissue having high, medium, low values of other physiological parameters such as, but not limited to, HbCO, HbMet, COHb, MetHb, and tHb. In some embodiments, the quality check insert 102 will have a body that will absorb and attenuate light resulting in values that are not related to physiological parameters. The non-physiological parameters can also have high, medium, or low values. The physiological and/or non-physiological parameter values that are expected when the quality check insert 102 absorbs and attenuates one or more predetermined wavelengths of light can be called verification data.

In some embodiments, the quality check inserts 102 will have a broader and more nuanced range of values not limited to high-high, high, medium-high, medium, medium-low, low, low-low and/or the like. Further, the use of high, medium, and low terminology is for discussion purposes only and not limiting. The quality check inserts 102 can be labeled with a numerical value and/or range to indicate the verification data to be expected with one or more predetermined wavelengths of light. Thus, the verification data can be a single value, a range, or a combination. The bodies 104, 106, 108 can include water and additional light absorbing constituents. In certain embodiments, the bodies of 104, 106, 108 can be a solid opaque, semi-opaque, and/or clear material.

The high values 104, medium values 106, and low values 108 quality check inserts 102 can be provided in a kit 101. The high values 104, medium values 106, and low values 108 of the quality check inserts 102 can have different color indicators for identification. For example, the high value, medium value, and low value quality check inserts 102 can also have different color caps 114, 116, 118, respectively, to identify the high, medium, and low values. In certain embodiments, the bodies 104, 106, 108 can have different colors from the caps 114, 116, 118 to provide a further nuanced method of identifying value of light attenuation of the quality check insert 102. For example, the high-high value quality check insert 102 can have a body 104 that is red and a cap 114 that is red. Continuing with the example, a medium-high value quality check insert 102 can have a body 104 that is red and a cap 114 that is yellow. A medium (or medium-medium) value quality check insert 102 can have a body 104 that is yellow and a cap 114 that is yellow.

In some embodiments, the bodies 104, 106, 108 and/or caps 114, 116, 118 will have symbol indicators signifying the verification data. The symbols can be scoring such as numerals I, II, III representing low, medium, and high values, respectively. Similarly, the symbols can be a range of asterisks, stars, or different shapes representing the values of verification data.

Light absorbing constituents as described above can be included within the bodies 104, 106, 108 and/or can be enclosed with caps 114, 116, 118. The caps 114, 116, 118 can be made from any suitable material, such as, but not limited to silicone, nylon, polyolefin, polystyrene, polyester, polypropylene, polyethylene rubber, vinyl, plastic, and/or the like.

Referring to FIG. 9, field data is represented by the readouts 915 on the display 910 that would be displayed during normal operation of the patient monitor 900 measuring and calculating the physiological parameters of a patient. The field data can vary depending on the calibration and proper functionality of the emitters, detectors, and/or patient monitor 900.

Once known, the field data can be compared to verification data. The verification data can be known values depending on the light absorbing constituents of a quality check insert 102 and predetermined wavelengths of light. When the quality check insert 102 is properly placed inside a sensor, and the emitters and detectors within the sensor are functioning properly, the field data should match the verification data.

In one embodiment, the verification data is written and associated with a particular quality check insert. For example, the kit 101 described for FIG. 1 can have a manual that provides the verification data associated with each of the quality check inserts 102 inside the kit 101. Upon obtaining the field data, and operator manually compares the field data to the verification data. In other embodiments, the verification data may be printed on the insert itself.

In another embodiment, the comparison of the field data to the verification data is automatic. For example, the verification data 715 can be stored in the storage 714 of a patient monitor and displayed as verification data 913. In one embodiment, the verification data 913 can be uploaded to the patient monitor 900 at the manufacturer. In another embodiment, the verification data 715 can be uploaded from a memory device. The memory device can be, for example, a USB drive. Prior to the use of the quality check insert 102 with the patient monitor, the USB drive can be inserted into the proper port, such as the Micro USB connector 330 with a standard USB to Micro USB converter if necessary. In some embodiments, the verification data 715 can be uploaded from the Internet or a website when the patient monitor has a network interface 716. An operator can directly guide the patient monitor through the user interface 712 to the proper location on the Internet to download the verification data. Alternatively or in combination, the operator can navigate to the website using an auxiliary computer, such as a standard personal computer, that is interfaced with the patient monitor through the network interface 716. Upon navigation to the proper website, the auxiliary computer downloads the verification data 715 from the website and uploads the verification data 715 to the storage 714 of the patient monitor. The patient monitor 900 can then display the verification data 913.

In some embodiments, the quality check insert 102 includes an information element. In one embodiment, the information element can be in electrical contact with the sensor. One advantage of using electrical contacts on a quality check insert 102 is that the patient monitor can recognize the absence of the information element and create an appropriate response indicating improper insertion or placement of the quality check insert 102. Upon electrical contact with the information element, the patient monitor can read the verification data that is stored on the information element or read its own memory when the data on the information element acts as an index to data stored on the monitor. The information element can be a passive device, such as a resistor, or an active circuit, such as a transistor network or memory chip. Upon reading the verification data, the patient monitor 900 can then display the verification data 913.

In another embodiment, the information element is a radio-frequency identification (RFID) chip. A patient monitor can emit radio waves to obtain verification data from the RFID without a physical electrical connection. Further detailed information about the configuration of an information element for an oximeter sensor and method for reading an information element with an attached oximeter sensor that can be used with a quality check insert is provided in U.S. Pat. No. 5,758,644 titled “Manual and Aromatic Probe Calibration” and U.S. Pat. No. 7,039,449 titled “Resposable Pulse Oximetry Sensor,” the disclosure of which is hereby incorporated by reference in its entirety. In embodiments where the patient monitor 900 performs an automatic comparison of the field data 915 to the verification data 913, the patient monitor 900 can have a visual and/or auditory indication/alarm when the patient monitor 900 is not functioning properly. In other embodiments, the patient monitor 900 may obtain the verification data via manufacturer upload, USB upload, Internet upload, information element, and/or RFID and display the verification data 913, while the operator performs a manual comparison to the field data 915.

FIGS. 10A-C illustrate embodiments of the quality check insert 102 including light absorbing constituents as described above. The quality check insert 102 can be a clear, semi-opaque, and/or opaque. FIG. 10A illustrates an embodiment of a quality check insert 102 having an envelope 1004 including a medium 1008 with light absorbing constituents 1006 suspended therein. The envelope 1004 can be elastic to conform to the shape of a sensor. The envelope 1005 can have contours and features 603 of FIG. 6 to aid in the placement and retention of the quality check insert 102. The envelope 1004 can be made from any suitable material, such as, but not limited to silicone, nylon, polyolefin, polystyrene, polyester, polypropylene, polyethylene rubber, vinyl, plastic, and/or the like. The envelope 1004, light absorbing constituents 1006, and/or medium 1008 can have optical properties as described herein. In one embodiment, the envelope 1005, light absorbing constituents 1006, and/or medium 1008 having optical properties as described herein without agitation by an active pulse sensor. In another embodiment, the envelope 1004, light absorbing constituents 1006, and/or medium 1008 can exhibit optical properties as described herein when agitated by an active pulse sensor as described herein.

FIG. 10B illustrates another embodiment of a quality check insert 102 having a solid body 1010. The solid body 1010 can be elastic to conform to the shape of a sensor. The solid body 1010 can have contours and features 603 to aid in the placement and retention of the quality check insert 102. The solid body 1010 can be made from any suitable material, such as, but not limited to silicone, nylon, polyolefin, polystyrene, polyester, polypropylene, polyethylene rubber, vinyl, plastic, and/or the like. The light absorbing constituents 1012 can be permanently suspended (not moving) within the solid body 1010. The solid body 1010 and/or light absorbing constituents 1012 can have optical properties as described herein.

FIG. 10C illustrates yet another embodiment of a quality check insert 102 having a solid body 1014. The solid body 1014 can be elastic to conform to the shape of a sensor. The solid body 1014 can have contours and features 603 to aid in the placement and retention of the quality check insert 102. The solid body 1014 can be made from any suitable material, such as, but not limited to silicone, nylon, polyolefin, polystyrene, polyester, polypropylene, polyethylene rubber, vinyl, plastic, and/or the like. Channels or veins 1016 can be disposed within the solid body 1014. Within the channels 1016, light absorbing constituents 1018 can be suspended in a medium 1020. The solid body 1014, channels 1016, light absorbing constituents 1018, and/or medium 1020 can be of a composition having optical properties as described herein. The light absorbing constituents 1018 and medium 1020 can include in the channels 1016 to more closely resemble a patient digit.

FIGS. 11A-D illustrate an embodiment of a quality check insert having a solid body. The quality check insert 1102 can be extruded into a mold to include certain features as described herein. The quality check insert 1102 can be fabricated using any other suitable or known process or processes, including injection molding, compression molding, thermoforming techniques, 3-D printing, and/or the like. The quality check insert 1102 can be made from any suitable material such as, but not limited to silicone, nylon, polyolefin, polystyrene, polyester, polypropylene, polyethylene rubber, vinyl, plastic, and/or the like. The body of the quality check insert 1102 can itself attenuate light having optical properties as described herein. The quality check insert 1102 can include features to help position and retain the quality check insert 1102 in an optical sensor such as the embodiment illustrated in FIG. 6.

FIG. 11A illustrates a top-front perspective view of an embodiment of the quality check insert 1102. The quality check insert 1102 can have a fingernail type stop 1104 that bumps up against a stop in an optical sensor. The stop 1104 can help prevent inserting the quality check insert 1102 too far into the optical sensor. The stop 1104 may include a semi rigid nail surface. The quality check insert 1102 can also have an emitter outline 1106 for assisting a user in an approximate placement in an optical sensor.

FIG. 12 illustrates a cutaway view of an embodiment of an optical sensor. As the user positions the quality check insert 1102, the emitter outline 1106 can be lined up with a transparent cover 1204 of a housing 1206 for the emitters 1208 included in an emitter shell 1210 of the optical sensor 1202. In some embodiments, the emitter outline 1106 can be a square-shaped or any other shaped indentation and/or protrusion on the body of the quality check insert 1102 similarly sized as the transparent cover 1204. In some embodiments, the emitter outline 1106 can be a line indentation and/or protrusion outline of a shape such as, for example, a line, square, rectangle, or the like.

In some embodiments, the quality check insert 1102 can include ridges or channels 1108 that engage and/or mate with an optical sensor. The quality check insert 1102 can include flaps 1110. The flaps 1110 can help to position and help the retention of the quality check insert 1102 in an optical sensor. For example, referring to FIG. 12, upon placement of the quality check insert 1102 into the clothespin style optical sensor 1202 and closure of the emitter shell 1210 and detector shell 1212, the flanges 1214 of the emitter shell 1210 can press against the flaps 1110. Pressure against the flaps 1110 can help secure the quality check insert 1102. The flaps 1110 can be compressed by the flanges 1214 and/or wrap either on the inside or outside walls of the flanges 1214.

FIG. 11B illustrates a top-back perspective view of an embodiment of the quality check insert 1102. The quality check insert 1102 can have a knob 1112. The knob 1112 can be used to hold the quality check insert 1102 during placement and alignment of the quality check insert 1102 in an optical sensor.

FIG. 11C illustrates a bottom-front perspective view of an embodiment of the quality check insert 1102. The back of the quality check insert 1102 can include ridges or channels 1114 that engage and/or mate with an optical sensor as discussed for FIG. 6. The quality check insert 1102 can have an indentation 1116 that align, engages and/or mates with a bump of a detector shell. The indentation 1116 can substantially mirror a bump 1216 such that the indentation 1116 can align, engage and/or mate with the bump 1216. The bump 1216 can house detectors. In some embodiments, the quality check insert 1102 has both an emitter outline 1106 and an indentation 1116, which upon proper placement of the quality check insert 1102, can help achieve the desired light path lengths between the emitters and detectors as described herein.

The quality check insert 1102 can have other features that help positioning and retention in an optical sensor. For example, the quality check insert 1102 can have bumpers 1118. The bumpers 1118 can act similarly to a stop 1104 as described herein. The bumpers 1118 can bump against certain features of the emitter shell 1210 and/or detectors shell 1212 upon insertion of the quality check insert 1102. Thus, the bumpers 1118 can help position the quality check insert 1102 such as, for example, preventing insertion of the quality check insert 1102 too far into, skewing to the right or left relative to, and/or rotating relative to the optical sensor. The bumpers 1118 can help retain structural integrity and/or desired shape of the quality check insert 1102 such as, for example, when a clothespin type sensor compresses the quality check insert. In another example, the bumpers 1118 can help retain structural integrity and/or desired shape when the flanges 1214 press against or compress the flaps 1110. In some embodiments, the bumpers 1118 can help retain structural integrity and/or desired shape by pressing against certain features of the optical sensor when a force is applied against the quality check insert 1102 and/or the flaps 1118. In some embodiments, the bumpers 1118 do not press against features of the optical sensor and help independently retain the structural integrity of the quality check insert 1102.

FIG. 11D illustrates a bottom-back perspective view of an embodiment of the quality check insert 1102. FIG. 11D illustrates the features of a flap 1110, knob 1112, ridges or channels 1114, indentation 1116, and bumper 1118 of a quality check insert as discussed herein.

FIG. 13 is a block diagram of an embodiment of a quality control system for quality control testing a patient monitoring system as discussed herein. In the embodiment illustrated in FIG. 13, data is gathered using a sensor and active pulse signals are processed, and parameters are calculated. While the below embodiments are described in referenced to plethysmograph waveforms (“pleths”), other waveforms, for example, not correlated to physiological parameters as discussed herein, can be incorporate into the quality control testing discussed herein.

In the embodiment illustrated in FIG. 13, step 1302 is executed. Step 1302 includes data acquisition, which may be obtained using an optical pulse oximetry sensor. Data is acquired using optical detectors. The data acquisition step is performed over a period of time. In an embodiment, one or more optical emitters and one or more optical detectors may be used to acquire data. In embodiments with multiple optical emitters, it may be beneficial to emit light using one emitter at a time. Emitting light with one emitter at a time can be used by the quality control system to determine which, if any, of the emitters are malfunctioning as described herein. The optical emitters can be turned on in series over time. If the optical emitters are used individually, the signal produced by the detectors may be time-shifted to obtain time-aligned data. In an embodiment, the data acquisition step may take approximately two minutes. A calibration signal may be obtained. The calibration signal may include data representing the gain of the optical sensor circuit and the current of the optical sensor circuit. The calibration data may include data representing demultiplexed output data, lowspeed demodulated data, current, gain, acceleration, and temperature. The acquired data may be organized by sample and associated with a frequency or time domain.

After the data acquisition step 1302, the data may be processed. In an embodiment, at step 1304, a logarithm can be taken of the output data collection signal (e.g., demodulated data) and, at step 1306, can be inputted through a broad bandpass filter (and/or other signal processing device or system used to process a narrow signal range) to obtain a normalized pleth. In an embodiment, the frequency range of the broad bandpass filter may be between 0.5 Hz and 8 Hz. The broad bandpass range of 0.5 Hz to 8 Hz may be selected to include normal pulse rate ranges as well as second and third harmonic signals in the filtered data. If the data signal is offset from zero, the offset may be removed to obtain a signal with a mean pleth of approximately zero. At step 1308, a standard deviation is calculated of the normalized pleth to obtain a noise level signal indicative of an overall noise level of the patient monitoring system. At step 1328, the noise level signal is limit tested between, for example, a predetermined upper noise value and a predetermined lower noise value. If the noise level signal falls between the upper and lower noise values, then the patient monitor may be deemed to have passed quality control and be functioning properly. In some embodiments, the noise level signal may be limit tested against only an upper predetermined noise value where, for example, a very low level noise level signal does not cause the patient monitor to fail quality control testing.

In an embodiment, at step 1310, the output data collection signal (e.g., demodulated data) can be inputted through a broad bandpass filter to obtain a DC signal. In an embodiment, the frequency range of the broad bandpass filter (and/or other signal processing device or system used to process a narrow signal range) may be between 0 Hz and 8 Hz. At step 1312, the data streams corresponding to the output data can be averaged. At step 1314, the DC signal may be divided by a gain characteristic of the system. The gain characteristic may be acquired as described in the data acquisition step above. In some embodiments, the gain characteristic can be based on the PGA described herein. At step 1316, the DC signal may further be divided by a power characteristic of the system. Power may be calculated using centroid interpolation using optical emitter temperature and current characteristics determined in manufacturing or by testing. At step 1318, a logarithm can be taken of the modified DC signal to obtain a transmittance characteristic signal. At step 1328, the transmittance characteristic signal is limit tested between, for example, a predetermined upper transmittance value and a predetermined lower transmittance value. If the transmittance signal value falls between the upper and lower transmittance values, then the patient monitor may be deemed to have passed quality control and be functioning properly. The upper and lower transmittance values can correspond to an absorbance range of a quality check insert as discussed herein, and in particular, in reference to FIG. 8.

In an embodiment, at step 1320, the output data collection signal (e.g., demodulated data) can be inputted through a broad bandpass filter (and/or other signal processing device or system used to process a narrow signal range) to obtain an active pulse normalized pleth measurement (npap) signal. In an embodiment, e.g., the band pass filter may pass through frequencies in a 1 Hz range around a center frequency pass through of 12.74 Hz on a 905 nanometer wavelength light emitting diode (LED). The bandpass filter can output data representing an active pulse normalized pleth measurement (npap). The normalized pleth (np) can be used as an input for numerical analysis. In an embodiment, the npap can be demodulated and input to a real-time pulse rate processing system. At step 1322, frequency analysis may be performed. The frequency analysis may include analysis of the fast Fourier transform (FFT), a discrete Fourier transform (which may use a FFT algorithm), or other frequency domain analysis techniques. The signal may be analyzed for strength and locations of harmonics. A logarithm may be taken of the signal. Frequency analysis can output a motor frequency signal corresponding to the rotation frequency of the active pulse motor. At step 1328, the motor frequency signal is limit tested between, for example, a predetermined upper frequency value and a predetermined lower frequency value. If the noise level signal falls between the upper and lower frequency values, then the patient monitor, and in particular the active pulse motor, may be deemed to have passed quality control and be functioning properly as discussed herein.

After the data acquisition step 1302, current signal data (current level signal) 1324 of the acquired data can be limit tested. Current level data can represent the electric current draw of light emitters of an optical sensor (e.g., current signal data for each emitter of the optical sensor) to generate a desired level of light intensity. At step 1328, the current level signal is limit tested between, for example, a predetermined upper current value and a predetermined lower current value. If the current level signal value falls between the upper and lower current values, the patient monitor, an in particular the light emitters, may be deemed to have passed quality control and be functioning properly. Current level signal limit testing may indicate, for example, a faulty light emitter (e.g., a burnt out or half burnt out light emitter). For example, if a desired light intensity level (e.g., by achieving a desired predetermined transmittance) is achieved (or not achieved) with an electric current draw by the light emitter being above the predetermined upper current value, the light emitter may be faulty.

After the data acquisition step 1302, gain signal data (gain level signal) 1326 of the acquired data can be limit tested. Gain level data can represent an amplification level of a detector signal from a detector of an optical sensor (e.g., gain level data for each detector of the optical sensor) to generate a desired level of signal intensity. At step 1328, the gain level signal is limit tested between, for example, a predetermined upper gain value and a predetermined lower gain value. If the gain level signal value falls between the upper and lower gain values, then the patient monitor, and in particular the detectors, may be deemed to have passed quality control and be functioning properly. Gain level signal limit testing may indicate, for example, a faulty light detector. For example, if a desired level of signal intensity is achieved (or not achieved) with the gain level of the detector signal above the predetermined upper gain value, the detector may be faulty.

In an embodiment, at step 1329, an acceleration signal from accelerometers of an optical sensor (e.g., an acceleration signal for each accelerometer of the optical sensor) is averaged and can be used for pleth validation. Acceleration criteria evaluate the presence of motion during a normalized pleth for quality control. Acceleration criteria may use various properties of the signal, e.g., amplitude, to determine the amount of sensor motion that occurred during data collection. A normalized pleth taken when too much motion occurred may not meet the acceleration criteria during quality control. At step 1328, the average acceleration signal is limit tested between, for example, a predetermined upper acceleration value and a predetermined lower acceleration value. If the acceleration signal value falls between the upper and lower acceleration values, then the patient monitor may be deemed to have passed quality control and be functioning properly. If a fail result for quality control testing is obtained, but motion has not occurred during the data acquisition, the optical sensor may have faulty accelerometers.

In an embodiment, at step 1330, a temperature signal (e.g., a temperature signal for each temperature sensor of the patient monitoring system) is averaged and can be used for pleth validation. Temperature criteria evaluate the ambient temperatures under which quality control is being performed. In some embodiments, the patient monitor and optical sensor should be used in a temperature range of about 5 to about 40 degrees ° C. At step 1328, the average temperature signal is limit tested between, for example, a predetermined upper temperature value and a predetermined lower temperature value. If the temperature signal value falls between the upper and lower temperature values, then the patient monitor may be deemed to have passed quality control and be functioning properly. If a fail result for quality control testing is obtained, but the ambient temperature is known to be within the upper and lower temperature values during the data acquisition, the patient monitoring system may have faulty temperature sensors.

In some embodiments, quality control parameters of a noise level signal, a transmittance characteristic signal, a motor frequency signal, a current level signal, a gain level signal, an acceleration signal, and/or a temperature signal as discussed herein may be used to determine if a patient monitor passes quality control. In some embodiments, any number or combination of quality control parameters as discussed herein may be used to determine if a patient monitoring system passes quality control. At step 1332, a quality control pass fail result can be generated (e.g., a visual or audible indicator as discussed herein) and can include a corresponding error code that can communicate (e.g., electronically, visually and/or audibly) one or more parameters that passed and/or failed.

### Additional Embodiments

In some embodiments, the bodies 104, 106, 108 and/or caps 114, 116, 118 can also indicate and/or correspond to a size of the quality check insert 102. In some embodiment, noninvasive sensors 201, 304, 404, 501, 901, 1202 as discussed herein may be different sizes to correspond to different sizes of measuring sites. For example, a noninvasive sensor 201, 304, 404, 501, 901, 1202 may be sized to accept, mate, and/or engage an adult's finger. A noninvasive sensor 201, 304, 404, 501, 901, 1202 may be sized to accept, mate, and/or engage a child's finger. A noninvasive sensor 201, 304, 404, 501, 901, 1202 may be sized to accept, mate, and/or engage an adult arm, child arm, and/or toddler arm. A noninvasive sensor 201, 304, 404, 501, 901, 1202 may be sized to accept, mate, and/or engage an adult's ear lobe, a child's ear lobe, a toddler's ear lobe, and/or other adult/child tissue measuring sites. The quality check insert 102 can be sized to be inserted into and/or accepted by a predetermined size of the noninvasive sensor. For example, the body 104 and/or cap 114 of a quality check insert 102 can indicate and/or correspond to a large (e.g., adult) size quality check insert 102 (large size relative to other quality check inserts 102). The quality check insert 102 can have a large body 104 to provide quality control testing for a large (e.g., adult) size noninvasive sensor. The body 106 and/or cap 116 of a quality check insert 102 can indicate and/or correspond to a medium (e.g., child) size quality check insert 102 (medium size relative to other quality check inserts 102). The quality check insert 102 can have a medium body 106 to provide quality control testing for a medium (e.g., child) size noninvasive sensor. The body 108 and/or cap 118 of a quality check insert 102 can indicate a small (e.g., toddler) size quality check insert 102 (small size relative to other quality check inserts 102). The quality check insert 102 can have a small body 108 to provide quality control testing for a small (e.g., toddler) size noninvasive sensor.

In some embodiments, the large 104, medium 106, and/or small 108 size bodies can have different absorption versus wavelength profiles (e.g., light absorption/transmittance values or range of values) corresponding to high, medium, low, etc. as discussed herein, including the absorption profile illustrated in FIG. 8. In some embodiments, the large 104, medium 106, and/or small 108 size bodies can have substantially the same absorption versus wavelength profiles (e.g., light absorption/transmittance values or range of values), for example, as illustrated in FIG. 8.

In some embodiments, the transmittance T is characterized by the equation:

$$T = ln ( \frac{{DC}_{average}}{Gain * Power} )$$

Power may be calculated using centroid interpolation using optical emitter temperature and current characteristics determined in manufacturing or by testing. The DC_{average} may be calculated using the indices calculated by finding the zero crossing of the demodulated or processing signal. The indices may be applied to the output data collection signal, including the DC offset. The DC_{average} represents an average value per pleth taken between the zero crossing indices as applied to the output data collection signal or other signal with a DC offset. The gain may be obtained during calibration.

In some embodiments, the frequency analysis may include a FFT performed on the input stream (np). In an embodiment, the FFT may be performed on the input stream associated with the 905 nm wavelength, since the 905 nm wavelength may have characteristics, including a high amplitude and/or a strong signal, that make the 905 nm wavelength useful for frequency analysis. The normalized pleth is summed over all input streams associated with the 905 wavelength, producing a summed normalized pleth npₛᵤₘ. FFTs may be performed on npₛᵤₘ by dividing the npₛᵤₘ signal into a number of samples. An FFT is then performed on a subset of the samples. Additional FFTs may be performed on subsets of samples until all the samples have been analyzed. In an embodiment, for example, a npₛᵤₘ signal may be divided into 2048 samples. An FFT may be performed on a first sample subset of the first 512 samples of the npₛᵤₘ signal, corresponding to a sample range between sample 0 and sample 512. An additional FFT may be performed on a subset of 512 samples shifted by 128 samples, corresponding to a sample range between sample 128 and sample 640. Additional FFTs may be performed, each FFT using the same number of samples as an input, 512 samples, and each FFT sampling a range shifted by 128 samples, until all the samples have been analyzed. For each FFT, local maximums may be detected. Each FFT may include multiple local maxima, corresponding to the pleth frequency and the harmonics of the pleth frequency. Comparing the amplitude of the local maxima may assist in determining the location of first-order and harmonic signals. A logarithm may be taken of the signal.

### CONCLUSION

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. Terms such as “a” or “an” can mean more than one instance of the feature for a particular embodiment. Further, using “one or more”, or similar terms such as “at least one”, for some features does not preclude “a” or “an” from also encompassing more than one.

Depending on the embodiment, certain acts, events, or functions of any of the methods described herein can be performed in a different sequence, can be added, merged, or left out altogether (e.g., not all described acts or events are necessary for the practice of the method). Moreover, in certain embodiments, acts or events can be performed concurrently, e.g., through multi-threaded processing, interrupt processing, or multiple processors or processor cores, rather than sequentially.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The blocks of the methods and algorithms described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, a hard disk, a removable disk, a CD-ROM, or any other form of computer-readable storage medium known in the art. An exemplary storage medium is coupled to a processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments of the disclosures described herein can be embodied within a form that does not provide all of the features and benefits set forth herein, as some features can be used or practiced separately from others. The scope of certain disclosures disclosed herein is indicated by the appended claims rather than by the foregoing description. All changes which come within the meaning and range of equivalency of the claims are to be embraced within their scope.

What is claimed is:

1. A quality control system comprising:
a noninvasive optical sensor configured to detect light attenuated by body tissue of a patient and output a detector signal responsive to the detected light;
a patient monitor configured to process the detector signal to determine measurement values for one or more physiological parameters of the patient; and
an insert shaped generally to mechanically mate with surfaces of the optical sensor, the insert having optical properties, wherein when the insert is properly placed within the sensor and irradiated by the sensor, the monitor processes detector signals, wherein the patient monitor provides display indicia indicative of whether the processed detector signals generate values within a predetermined range of values, the predetermined range associated with the optical properties of the insert.

2. The quality control system of claim 1, wherein the insert has at least one of a color indicator or a symbol indicator corresponding to verification data.

3. The quality control system of claim 2, wherein the verification data comprises ranges of data.

4. The quality control system of claim 1, wherein the optical properties of the insert are at least in part due to light absorbing constituents suspended within a body of the insert.

5. The quality control system of claim 1, wherein the display indicia includes indicia indicating a quality pass or fail.

6. The quality control system of claim 1, wherein the processing by the monitor comprises processing detector signals when the insert is placed in the sensor is similar to processing detector signals when tissue is placed in the sensor.

7. A quality control system comprising:
a noninvasive optical sensor configured to detect light attenuated by body tissue of a patient and output a detector signal responsive to the detected light;
a patient monitor configured to process the detector signal to determine measurement values for one or more physiological parameters of the patient; and
an insert shaped generally to engage with surfaces of the optical sensor, the insert having optical properties, wherein when the insert is properly placed within the sensor and attenuates light emitted by the sensor, the monitor processes detector signals, wherein the patient monitor determines whether a processed detector signal generates a transmittance value within a predetermined transmittance range of values, the predetermined transmittance range associated with the optical properties of the insert.

8. The quality control system of claim 7, wherein the patient monitor generates at least one of a visual indicia or an audible indicia indicating a quality pass or fail.

9. The quality control system of claim 7, wherein when the insert is properly placed within the sensor and attenuates light emitted by the sensor, the patient monitor determines whether an electric current draw of one or more light emitters of the sensor to generate a desired level of light intensity is within a predetermined current range of values, and wherein when the electric current draw is not within the predetermined current range of values, at least one of the light emitters of the sensor is determined to have failed.

10. The quality control system of claim 7, wherein when the insert is properly placed within the sensor and attenuates light emitted by the sensor, the monitor processes the detector signal, wherein the patient monitor determines whether a gain level of the detector signal to generate a desired level of signal intensity is within a predetermined gain range of values, wherein the detector signal is associated with one or more detectors of the sensor, and wherein when the gain level is not within the predetermined gain range of values, at least one of the detectors of the sensor is determined to have failed.

11. The quality control system of claim 7, wherein when the insert is properly placed within the sensor, the patient monitor determines whether a rotation frequency of an active pulse motor of the sensor is within a predetermined frequency range of values, and wherein when the rotation frequency of the active pulse motor is not within the predetermined frequency range of values, the active pulse motor is determined to have failed.

12. The quality control system of claim 7, wherein the monitor processes the detector signals, and wherein the patient monitor determines whether a noise level associated with the detector signals is within a predetermined noise level range of values.

13. The quality control system of claim 7, wherein the patient monitor determines whether values generated by an acceleration signal associated with an accelerometer of the sensor are within a predetermined acceleration range of values, and wherein when the sensor is not moved and the values generated by the acceleration signal associated with the accelerometer are not within the predetermined acceleration range of values, the accelerometer is determined to have failed.

14. The quality control system of claim 7, wherein the patient monitor determines whether values generated by a temperature signal associated with a temperature sensor of the sensor are within a predetermined temperature range of values, and wherein when ambient temperature is within an ambient temperature range corresponding to the predetermined temperature range of values and the values generated by the temperature signal associated with the temperatures sensor are not within the predetermined temperature range of values, the temperature sensor is determined to have failed.

15. A quality control insert for quality control testing of a noninvasive patient monitor, the quality control insert comprising:
a body comprising light absorbing constituents having optical properties, the body configured to mate with a noninvasive optical sensor of a patient monitor configured to determine one or more physiological parameters of a patient,
wherein the optical properties are associated with the light absorbing constituents attenuating light at predetermined light absorption values based on wavelengths of the light when the body of the insert is irradiated by the sensor.

16. The quality control insert of claim 15, wherein the light absorbing constituents are suspended in the body of the insert.

17. The quality control insert of claim 15, wherein the body of the insert comprises features configured to place the body in the sensor at a predetermined position and help retain the body in the sensor at the predetermined position.

18. The quality control insert of claim 15, wherein the body comprises an emitter outline for aligning the emitter outline with emitters of the sensor.

19. The quality control insert of claim 15, further comprising bumpers configured to aid in positioning the body in the sensor at a predetermined position.

20. The quality control insert of claim 15, wherein the bumpers comprise a stop configured to inhibit insertion of the body into the sensor beyond the predetermined position

21. The quality control insert of claim 15, wherein the body comprises an indentation that mirrors a bump of the sensor, the bump housing light detectors of sensor, the indentation mating with the bump when the body is inserted into the sensor at a predetermined position.

22. The quality control insert of claim 15, wherein the body comprises a knob for holding the quality control insert during placement and alignment of the body in the sensor.
